(19) United States
(12) Patent Application Publication
(10) Pub. No.: US 2004/0039197 A1
(43) Pub. Date: Feb. 26, 2004

(54) Cyclic indole and heteroindole derivatives and methods for making and using as pharmaceuticals

(76) Inventors: Heinz Weinberger, Kelkheim (DE); Thomas Beckers, Frankfurt a.M. (DE); Mathias Schmidt, Konstanz (DE); Silke Baasner, Schoneck (DE); Bernd Nickel, Muhltal (DE)

Correspondence Address: GABRIEL P. KATONA, GOODWIN PROCTER L.L.P., 599 LEXINGTON AVENUE, 40TH FLOOR, NEW YORK, NY, 10022, US

(21) Appl. No.: 10/233,135

(22) Filed: Aug. 30, 2002

Related U.S. Application Data
(60) Provisional application No. 60/317,102, filed on Sep. 4, 2001.

(51) Int. Cl. C07D 498/02; C07D 491/02; C07D 471/02

(52) U.S. Cl. 544/9; 544/66; 544/184; 544/251; 546/81; 548/208; 548/217; 548/150; 548/262.4

(57) ABSTRACT

The invention relates to novel, substituted, fused indole and heteroindole derivatives of the general formula I

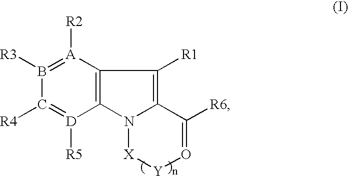

their tautomers, stereoisomers, mixtures and pharmaceutically acceptable salts, their synthesis and their use as pharmaceuticals, especially as anti-tumor agents, for mammals, especially for man.

The invention relates to new, substituent indole and heteroindole derivatives of the general formula

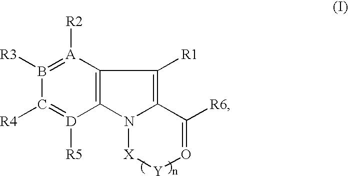

their tautomers, their stereoisomers, their mixtures and their pharmaceutically acceptable salts, their synthesis and their use as pharmaceuticals, especially as anti-tumor agents in mammas, particularly in man.

The German patent application of 28-04-2000 (Patent ASTA Medica AG with Priv.-Doz. Dr. Mahboobi) discloses a method for the synthesis of 2-acyl indoles by way of the corresponding 2-lithium indoles.

In the publication by Theophil Eicher and Ralph Rohde, Synthesis 1985, Pages 619-625, the synthesis of 1,2-diphenyl-3a-aza-cyclopenta[a]lindene-3-one is described. A medical use of said compound is neither disclosed or suggested.

According to one aspect of the invention, compounds of the general formula 1

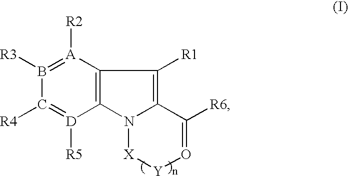

in which

R1 represents hydrogen, unsubstituted or fully or partly substituted, identically or differently, (C6-C14)-aryl, unsubstituted or fully or partly substituted, identically or differently, (C1-C13)-heteroaryl, having at least one to four N, NH, O and/or S as ring elements, unsubstituted or fully or partly substituted, identically or differently (C3-C8)-cycloalkyl, or unsubstituted or fully or partly substituted, identically or differently (C1-C20)-alkyl,

A, B, C, or D, independently of one another, represents a carbon atom or a nitrogen atom, substituted with R2-R5,

R2, R3, R4 and R5, independently of one another represent a free electron pair (when A, B, C or D represents nitrogen), hydrogen, halogen, cyano, nitro, hydroxy, linear or branched (C1-C6)-alkyl, linear or branched (C1-C6)-alkyl, substituted with one or more halogen atoms, linear or branched (C1-C6)-alkoxy, substituted with one or more halogen atoms, linear or branched (C1-C6)-alkoxy, linear or branched (C1-C6)-alkylenedioxy, (C1-C6)-alkylcarbonyloxy, (C1-C6)-alkoxycarbonyloxy, (C1-C6)-alkylthio, (C1-C6)-alkylsulfinyl, (C1-C6)-alkylsulfonyl, carboxy, carboxy (C1-C6)-alkyl ester, carboxamide, N—(C1-C6)-alkylcarboxamide, N,N-di-(C1-C6)-alkylcarboxamide, (C1-C6)-alkoxy-(C1-C6)-alkyl, amino, mono-(C1-C6)-alkylamino, di-(C1-C6)-alkylamino, the two (C1-C6) groups together being able to form a ring, which optionally has one or more NH, N—(C1-C6)-alkyl, O or S, (C6-C14)-aryl, (C6-C14)-aryloxy, (C6-C14)-aryl-(C1-C6)-alkyl, (C6-C14)-aryl-(C1-C6)-alkoxy-(C1-C6)-alkyl, (C1-C6)-alkylcarbonyl, (C1-C6)-alkoxycarbonyl, hydroxy, in which two directly adjacent groups can be linked to one another;

R6 represents unsubstituted or fully or partly substituted, identically or differently, (C6-C14)-aryl, unsubstituted or fully or partly substituted, identically or differently, (C1-C13)-heteroaryl, having at least one to four N, NH, O and/or S as ring elements, unsubstituted or fully or partly substituted, identically or differently, (C3-C8)-cycloaklyl, unsubstituted or fully or partly substituted, identically or differently, linear or branched (C1-C20)-alkyl, the identical or different substituents being selected from the group comprising hydrogen, fluorine, chlorine, bromine, iodine, cyano, nitro, hydroxy, (C1-C6)-alkyl, (C1-C6)-alkoxy, carboxy, (C1-C6)-alkyl, substituted, identically or differently, with one or more halogen atoms, (C1-C6)-alkoxy, substituted identically or differently with one or more halogen atoms, linear or branched (C2-C6)-alkenyl, linear or branched (C2-C6)-alkinyl, (C3-C8)-cycloalkyl, linear or branched (C1-C6)-alkoxy, linear or branched (C1-C6)-alkylenedioxy, (C1-C6)-alkoxy-(C1-C6)-alkyl, (C6-C14)-aryl, (C6-C14)-aryl-(C1-C6)-alkyl, (C6-C14)-aryl-(C1-C4)-alkoxy-(C1-C6)-alkyl;

X represents carbonyl-(C═O), sulfoxide-(S═O) or the sulfonyl group (SO₂);

Y represents an oxygen atom or a nitrogen atom (NR7), substituted by the R7 group, in which

R7 represents unsubstituted or fully or partly substituted, identically or differently, (C6-C14)-aryl, unsubstituted or fully or partly substituted, identically or differently, (C1-C13)-heteroaryl, having at least one to four N, NH, O and/or S as ring elements, (C3-C8)-cycloalkyl, unsubstituted or fully or partly substituted, identically or differently, (C1-C20)-alkyl, which may be linear or branched, wherein the identical or different substituents are selected from the group comprising hydrogen, fluorine, chlorine, bromine, iodine, cyano, nitro, hydroxy, (C1-C6)-alkyl, (C1-C6)-alkoxy, carboxy, (C1-C6)-alkyl, substituted identically or differently with one or more halogen atoms, (C1-C6)-alkoxy, substituted identically or differently with one or more halogen atoms, linear or branched (C2-C6)-alkenyl, linear or branched (C2-C6)-alkinyl, (C3-C8)-cycloalkyl, linear or branched (C1-C6)-alkoxy, linear or branched (C1-C6)-alkylenedioxy, (C1-C6)-alkoxy-(C1-C6)-alkyl, linear or branched mono-(C1-C6)-alkylamino, linear or branched di-(C1-C6)-alkylamino, wherein the two (C1-C4) groups together may form a ring, which optionally has one or more NH, N—(C1-C6)-alkyl, O or S, (C6-C14)-aryl, (C6-C14)-aryl-(C1-C6)-alkyl, (C6-C14)-aryl-(C1-C4)-alkoxy-(C1-C6)-alkyl, (C1-C6)-alkylcarbonyl, (C1-C6)-alkoxycarbonyl;

n is 0 or 1, with the proviso that, when n=0,

Z represents a carbon atom (C—R8), substituted with the R8 group wherein

R8 represents unsubstituted or fully or partly substituted, identically or differently, (C6-C14)-aryl, unsubstituted or fully or partly substituted, identically or differently (C1-C13)-heteroaryl, which has at least one to four N, NH, O and/or S as ring elements, unsubstituted or fully or partly substituted, identically or differently, (C3-C8)-cycloalkyl, unsubstituted or fully or partly substituted, identically or differently, linear or branched, (C1-C20)-alkyl, the identical or different substituents being selected from the group comprising hydrogen, fluorine, chlorine, bromine, iodine, cyano, nitro, hydroxy, (C1-C6)-alkyl, (C1-C6)-alkoxy, carboxy, (C1-C6)-alkyl, which is substituted identically or differently by one or more halogen atoms, (C1-C6)-alkoxy, which is substituted identically or differently by one or more halogen atoms, linear or branched (C2-C6)-alkenyl, linear or branched (C2-C6)-alkinyl, (C3-C8)-cycloalkyl, linear or branched (C1-C6)-alkoxy, linear or branched (C1-C6)-alkylenedioxy, linear or branched (C1-C6)-alkylthio, (C1-C4)-alkylsulfinyl, (C1-C4)-alkylsulfonyl, (C6-C14)-arylthio, (C6-C14)-arylsulfinyl, (C6-C14)-arylsulfonyl, (C1-C6)-alkoxy-(C1-C6)-alkyl, linear or branched mono-(C1-C6)-alkylamino, linear or branched di-(C1-C6)-alkylamino, wherein the two (C1-C4) groups together can form a ring, which optionally has one or more NH, N—(C1-C6)-alkyl, O or S, (C6-C14)-aryl, (C6-C14)-aryloxy, (C6-C14)-aryl-(C1-C6)-alkyl, (C6-C14)-aryl-(C1-C4)-alkoxy-(C1-C6)-alkyl, (C1-C6)-alkylcarbonyl, (C1-C6)-alkoxycarbonyl, linear or branched mono-N—(C1-C6)-alkylcarbonylamino, linear or branched di-N,N-(C1-C6)-alkylcarbonylamino, linear or branched mono-N-(C1-C6)-alkoxycarbonylamino, linear or branched di-N,N-(C1-C6)-alkoxycarbonylamino, linear or branched N—(C1-C6)-alkylcarbonylamino-N—(C1-C6)-alkylamino, linear or branched N—(C1-C6)-alkoxycarbonylamino-N—(C1-C6)-alkylamino,

and, when n=1,

Z represents a nitrogen atom;

their tautomers, stereo isomers, mixtures and pharmaceutically tolerated salts.

Pursuant to a further aspect of the invention, the compounds are characterized by the fact that R1 represents hydrogen, R2, R3, R4 and R5 independently of one another represent hydrogen, halogen or (C1-C6)-alkoxy, R6 represents unsubstituted or fully or partly substituted, identically or differently, linear or branched (C1-C20)-alkyl or unsubstituted or fully or partly substituted, identically or differently, (C6-C14)-aryl, which is substituted with (C1-C6)-alkoxy and halogen, and Y represents oxygen or the N—R7 group, in which R7 represents unsubstituted or fully or partly substituted, identically or differently, (C6-C14)-aryl, X is carbonyl (C═O), Z is a nitrogen atom and n=1.

According to a further aspect of the invention, compounds, characterized in that R1 represents hydrogen, R2, R3, R4 and R5 independently of one another represent hydrogen, halogen or (C1-C6)-alkoxy, R6 represents unsubstituted or fully or partly substituted, identically or differently, linear or branched (C1-C20)-alkyl or unsubstituted or fully or partly substituted, identically or differently, (C1-C6)-alkoxy and halogen-substituted (C6-C14)-aryl, n=0, Z represents the C—R8 group, in which R8 represents unsubstituted or fully or partly substituted, identically or differently, (C6-C14)-aryl, substituted with (C1-C6)-alkoxy and halogen, and X represents carbonyl (C═O), are made available.

According to a further aspect of the invention, the inventive compounds, named above, are used as pharmaceuticals.

According to a further aspect of the invention, the use of one of the inventive compounds, named above, for controlling tumor diseases in mammals, especially in man, is made available.

According to a further aspect of the invention, pharmaceuticals, containing at least one of the inventive compounds named above, together with adjuvants, diluents and/or carriers, are made available.

In accordance with a further aspect of the invention, a method for the synthesis of compounds of the general formula I

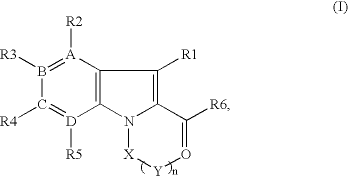

wherein A, B, C, D, R1, R2, R3, R4, R5, R6, X, Y, Z and n have the meanings given above, characterized by the reaction of the ketone of the general formula

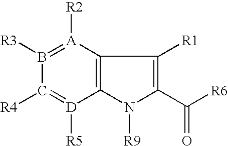

wherein A, B, C, D, R1, R2, R3, R4, R5 and R6 have the meanings given above and R9 represents hydrogen or a suitable leaving group, such as unsubstituted or fully or partly substituted, identically or differently, linear or branched (C1-C6)-alkylcarbonyl, linear or branched (C1-C6)-alkoxycarbonyl, substituted (C6-C14)-aryl-(C1)-alkyl, linear or branched (C1-C6)-alkylsulfonyl and (C6-C14)-arylsulfonyl, which is unsubstituted or fully substituted with (C1-C6)-alkyl,

1.) (if n=1) with

1.1) (if Y=oxygen) hydroxylamine or

1.2) (if Y═NR7), a hydrazine derivative H2N—NH—R7, wherein R7 as the meaning given above and reaction of the product, so obtained, with an activated carbonyl, sulfoxide or sulfonyl derivative with ring closure and

or

2.) (if n=0) with a phenylacetic acid derivative X1-CO—CH2-R8, wherein X1 represents a suitable leaving group, such as halogen or (C1-C6)-alkoxy and R8 has the meaning given above,

and subsequent ring closure in the presence of a base, is made available.

The inventive compounds of the general formula I can be obtained by known methods, the steps of which are known. For example, the steps, described below, are suitable:

a) reaction of an indole or heteroindole compound, which optionally is provided with a suitable nucleophilic leaving group, with organometallic compounds:

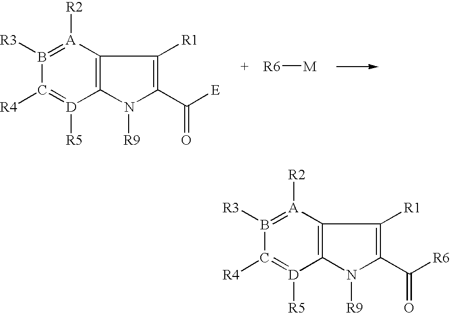

in which

R1, R2, R3, R4, R5, R6, A, B, C and D are defined as above,

R9 represents hydrogen, linear or branched (C1-C6)-alkylcarbonyl, which is not substituted or substituted with one or more halogen atoms, linear or branched (C1-C6)-alkoxycarbonyl, substituted (C6-C14)-aryl-(C1)-alkyl, linear or branched (C1-C6)-alkylsulfonyl and (C6-C14)-arylsulfonyl, which is unsubstituted or fully substituted with (C1-C6)-alkyl,

E represents OH, a halogen atom, such as a fluorine, chlorine or bromine atom, (C1-C6)-alkoxy, imidazole and

M for Li, Mg—R10

wherein R10 is a halogen atom, such as a chlorine, bromine or iodine atom;

in the event that n=0

b1) reaction of the 2-acylindole or heteroindole compounds with a reagent, which is provided with a suitable nucleophilic leaving groups with optional simultaneous or previous removal of the R9 substituent at the indole nitrogen atom:

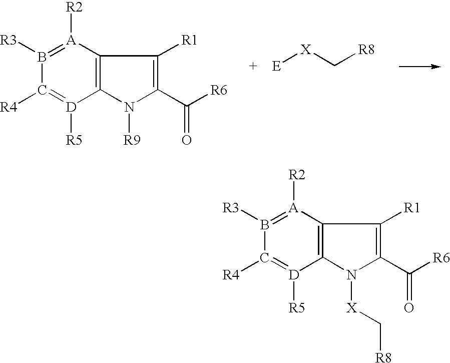

wherein

R1, R2, R3, R4, R5, R6, R8, R9, A, B, C, D, E and X are defined as above,

c1) reaction of the 2-acylindole or heteroindole compounds with base, preferably sodium hydride:

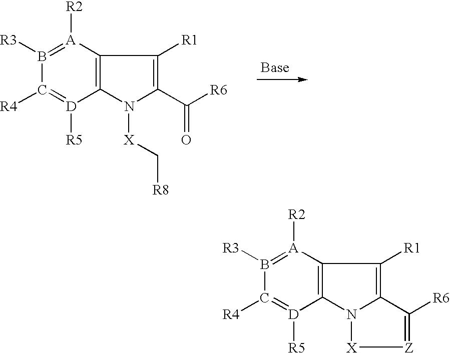

wherein

R1, R2, R3, R4, R5, R6, R8, A, B, C, D and X are defined as above and Z is a carbon atom, which is substituted with the R8 group,

And, in the event that n is 1:

b2) reaction of the 2-acyl-indole or heteroindole compounds with optionally substituted, primary amino derivatives:

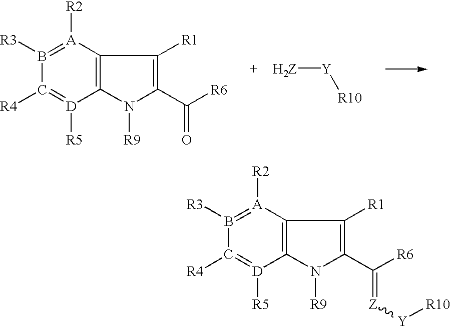

wherein

R1, R2, R3, R4, R5, R6, R9, A, B, C, D and Y are defined as above,

Z represents a nitrogen atom and

R10 represents hydrogen, linear or branched (C1-C6)-alkyl, substituted with one or more halogen atoms, linear or branched (C1-C6)-alkyl, substituted with one or more halogen atoms, linear or branched (C1-C6)-alkoxy, (C2-C6)-alkenyl, (C2-C6)-alkinyl, (C3-C8)-cycloalkyl, (C1-C6)-alkylcarbonyl, (C1-C6)-alkoxycarbonyl, (C1-C6)-alkylsulfinyl, (C1-C6)-alkylsulfonyl, (C1-C6)-alkoxy-(C1-C6)-alkyl, (C6-C14)-aryl, (C6-C14)-aryl-(C1-C6)-alkyl, (C6-C14)-aryl-(C1-C6)-alkoxy-(C1-C6)-alkyl, (C1-C6)-alkylcarbonyl, (C1-C6)-alkoxycarbonyl;

c2) reaction of the indole or heteroindole compounds with a reagent, having suitable nucleophilic leaving groups, with optional simultaneous or prior removal of the R9 substituted at the indole nitrogen atom:

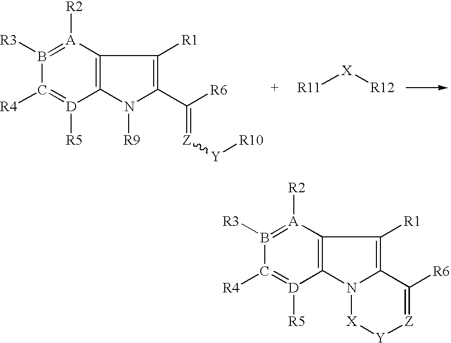

wherein

R1, R2, R3, R4, R5, R6, R9, A, B, C, D and X are defined as above, Z is a nitrogen atom and R10 represents a hydrogen atom,

and

R11 and R12, independently of one another, represent neucleophilic leaving groups, such as a halogen atom, such as a chlorine, bromine or iodine atom, (C1-C6)-alkoxy or imidazolide.

However, the synthesis is carried out particularly advantageously by reacting an isolated indole- or heteroindole carboxylic imidazolide, or one produced in situ, of the general formula II

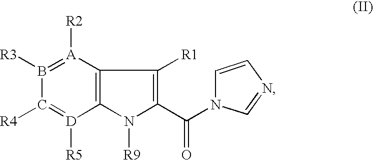

in which

R1, R2, R3, R4, R5, R9, A, B, C and D are defined as above, with Grignard reagents,

the indole or heteroindole of the general formula III

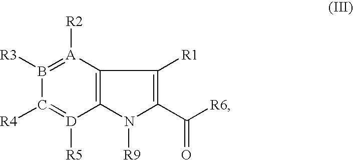

R1, R2, R3, R4, R5, R9, A, B, C and D are defined as above, with hydroxylamine,

the indole or heteroindole of the general formula IV

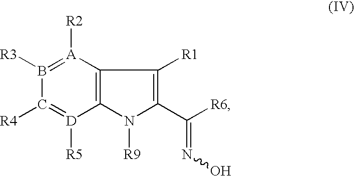

in which

R1, R2, R3, R4, R5, R6, A, B, C and D are defined as above, are cyclized with N,N′-carbonyl diimidazole to the indole or heteroindole derivatives of the general formula V

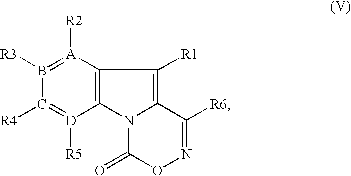

and

the indole or heteroindole derivatives of the above-mentioned formula III, in which R1, R2, R3, R4, R5, R6, A, B, C and D are defined as above and R9 is a hydrogen atom, are cyclized with optionally substituted phenylacetic acid halides in the presence of, for example, sodium hydride as base to the indole or heteroindole derivatives of the general formula VI

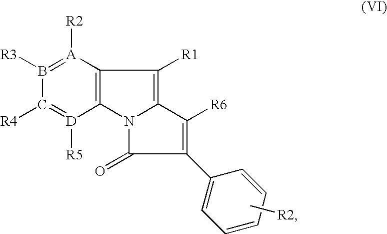

The compounds, which are used as starting materials and partly are commercially available or known in the literature, are obtained by methods known from the literature. Moreover, their synthesis is described in the examples. The methods, known from the literature, are described for example in L. and M. Fieser, Organische Chemie, 2 edition, 1979, pages 1417 to 1483, as well as in the references cited there on pages 1481-1483, Houben-Weyl-Müller, Methoden der organischen Chemie and Ullmanns Encyklopädie der technischen Chemie.

Furthermore, the compounds obtained, having the general formula I, can be separated into their enatiomers and/or diastereoisomers. For example, the compounds of the general formula I, which are obtained as racemates, can be separated by known methods into their optical isomers and compounds of the general formula I with at least 2 asymmetric carbon atoms can be separated on the basis of their physical and chemical differences by known methods, such as, chromatography and/or fractional crystallization into their diastereoisomers, which, in the event that they are obtained in the racemic form, can be separated, as mentioned above, into their enantiomers.

The enantiomeric separation is carried out preferably by a column chromatographic separation of chiral phases or by recrystallization from an optically active solvent or by reaction with an optically active substance, which forms salts or derivatives, such as esters or amides, with the racemic compound.

Moreover, the compounds of formula I, which are obtained, can be converted into their salts, especially, for pharmaceutical application, into their pharmacologically and physiologically compatible salts with inorganic or organic acids. As acids for this purpose, hydrobromic acid, sulfuric acid, phosphoric acid, fumaric acid, succinic acid, lactic acid, citric acid, tartaric acid or maleic acid come into consideration.

In addition, the compounds of formula I, in the event that they contain an acidic group, such as a carboxyl group, can be converted, if desired, into their salts with inorganic or organic basis, especially, for pharmaceutical use, into their physiologically compatible salts. As bases, sodium hydroxide, potassium hydroxide, cyclohexylamine, ethanolamine, diethanolamine and triethanolamine, for example, come into consideration.

The invention is to be explained in greater detail below by means of examples, without being limited to these examples.

General Method for the Synthesis of the Inventive 2-Acyl indoles

Method a) Isolation of the Imidazole-1-yl-(1H-indole-2-yl)-methanone and Subsequent Reaction with Organometallic Reagents

To a solution of 60 mmoles (11.47 g) of 5-methoxyindole-2-carboxylic acid in 200 mL of tetrahydrofuran, a solution of 72 mmoles (11.67 g, 1.2 equivalents), of N,N′-carbonyl diimidazole in 250 mL of tetrahydrofuran was added dropwise at room temperature with stirring within 60 minutes. After stirring for a further 15 minutes, the solvent was evaporated in a rotary evaporator and the residue recrystallized from 220 mL of a 3:2 mixture of tetrahydrofuran and hexane. Imidazole-1-yl-(5-methoxy-1H-indole-2-yl)-methanone was obtained as an orange brown solid with a melting point higher than 300° C. (dec.)

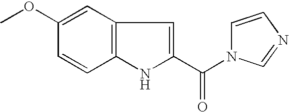

A solution of 1 equivalent of imidazole-1-yl-(5-methoxy-1H-indole-2-yl)-methanone in tetrahydrofuran (3 mL/mmole) was treated at 0° C. dropwise with 2.2 equivalents of the organometallic compound, so that the internal temperature did not exceed 5° C. The conversion during the reaction was followed by thin-layer chromatography, using a 1:1 mixture of ethyl acetate and hexane as solvent. At the end of the reaction, the reaction solution was mixed with water (10 mL/mmole) and adjusted to a pH of 6 with concentrated hydrochloric acid. The organic phase was removed and the aqueous phase was extracted three times with ethyl acetate (in each case, with 2 mL/mmole). After the combined organic phases were dried over magnesium sulfate, the solvent was evaporated in a rotary evaporator and the residue recrystallized from alcohol.

### EXAMPLE A1

Reagent A1: methyl magnesium chloride, 3.0 M Solution in tetrahydrofuran

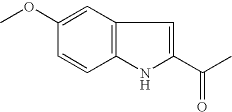

1-(5-methoxy-1H-indole-2-yl)-ethanone

Melting point: 164° C.-167° C. (2-propanol)

### EXAMPLE A2

Reagent A2: phenyl magnesium bromide, 3.0 M Solution in diethyl ether

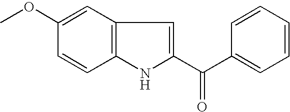

(5-methoxy-1H-indole-2-yl)-phenyl-methanone

Melting point: 164° C.-166° C. (n-butanol)

### EXAMPLE A3

Reagent A3: 3-methoxyphenyl magnesium bromide, 1.0 M Solution in tetrahydrofuran

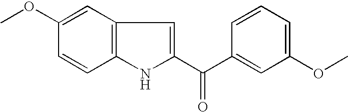

(5-methoxy-1H-indole-2-yl)-(3-methoxyphenyl)-methanone

Melting point: 143° C.-145° C. (n-butanol)

### EXAMPLE A4

Reagent A4: 4-methoxyphenyl magnesium bromide, 0.5 M Solution in tetrahydrofuran

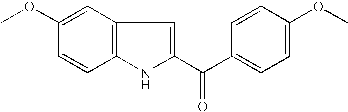

(5-methoxy-1H-indole-2-yl)-(4-methoxyphenyl)-methanone

Melting point: 155° C.-158° C. (n-butanol)

### EXAMPLE A5

Reagent A5: 4-chlorophenyl magnesium bromide, 1.0 M Solution in diethyl ether

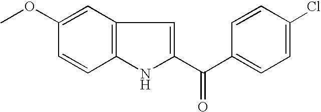

(4-chlorophenyl)-(5-methoxy-1H-indole-2-yl)-methanone

Melting point: 190° C.-192° C. (n-butanol)

### EXAMPLE A6

Reagent A6: 2-thienyl lithium, 1.0 M Solution in tetrahydrofuran

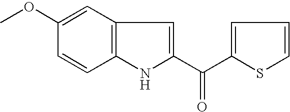

(5-methoxy-1H-indole-2-yl)-thiophene-2-yl-methanone

Melting point: 152° C.-154° C. (n-butanol)

Method B) One reactor variation: Synthesis of imidazole-1-yl-(1H-indole-2-yl)-methanone and Subsequent Reaction with Organometallic Reagents in situ

To a solution of 25 mmoles of indole-2-carboxylic acid in tetrahydrofuran (2 mL/mmole) a solution of 26 mmoles (1.05 equivalents) of N,N′-carbonyl diimidazole in tetrahydrofuran (3 mL/mmole) was added dropwise with stirring in an inert gas atmosphere at room temperature within 20 minutes. After being stirred for a further 60 minutes, the reaction solution was cooled to 0° C. and treated dropwise with the 3.5 equivalents of the organometallic compound, so that the internal temperature did not exceed 5° C. (about 60 minutes). The reaction was followed by means of thin-layer chromatography (with a 1:1 mixture of ethyl acetate and hexane as solvent). At the end of the reaction, the reaction solution was mixed with water (10 mL/mmole) and adjusted to a pH of 6 with concentrated hydrochloric acid. The organic phase was removed and the aqueous phase was extracted three times with ethyl acetate (in each case, with 2 mL/mmole). After the combined organic phases were dried over magnesium sulfate, the solvent was evaporated in a rotary evaporator and the residue recrystallized from alcohol.

### EXAMPLE B1

Educt: indole-2-carboxylic acid

Reagent B1: 2-methoxyphenyl magnesium bromide, 1.0 M Solution in tetrahydrofuran

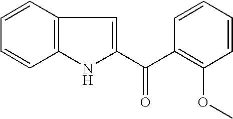

(1H-indole-2-yl)-(2-methoxyphenyl)-methanone

Melting point: 129° C.-130° C. (4:1 mixture of ethanol and water)

### EXAMPLE B2

Educt: indole-2-carboxylic acid

Reagent A3: 3-methoxyphenyl magnesium bromide, 1.0 M Solution in tetrahydrofuran

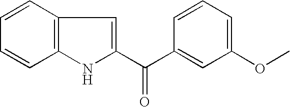

(1H-indole-2-yl)-(3-methoxyphenyl)-methanone

Melting point: 119° C.-121° C. (2-propanol)

### EXAMPLE B3

Educt: 5-methoxyindole-2-carboxylic acid

Reagent A1: methyl magnesium chloride, 3.0 M Solution in tetrahydrofuran

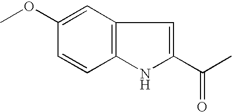

1-(5-methoxy-1H-indole-2-yl)-ethanone

Melting point: 164° C.-167° C. (2-propanol)

### EXAMPLE B4

Educt: 5-methoxyindole-2-carboxylic acid

Reagent B4: ethyl magnesium chloride, 3.0 M Solution in tetrahydrofuran

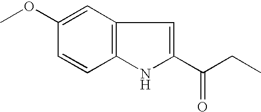

1-(5-methoxy-1H-indole-2-yl)-propane-1-one

Melting point: 173° C.-175° C. (2-propanol)

### EXAMPLE B5

Educt: 5-methoxyindole-2-carboxylic acid

Reagent A2: phenyl magnesium bromide, 3.0 M Solution in diethyl ether

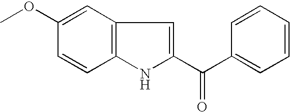

(5-methoxy-1H-indole-2-yl)-phenyl-methanone

Melting point: 164° C.-166° C. (n-butanol)

### EXAMPLE B6

Educt: 5-methoxyindole-2-carboxylic acid

Reagent A3: 3-methoxyphenyl magnesium bromide, 1.0 M Solution in tetrahydrofuran

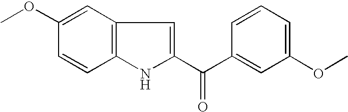

(5-methoxy-1H-indole-2-yl)-(3 -methoxyphenyl)-methanone

Melting point: 143° C.-145° C. (n-butanol)

General Method for the Synthesis of the Inventive Oxadiaza Derivatives

Method C) Synthesis, Isolation and Purification of indole-2-yl-oximes and Subsequent Reaction with N,N′-carbonyldiimidazole

C1) General Synthesis of indole-2-yl-oximes:

A suspension of 1 equivalent of the 2-acyl indole in ethanol (10 mL/mmole), prepared by method A or B, was treated with 1.5 equivalents of solid hydroxylamine hydrochloride and subsequently treated dropwise, with stirring, with 3.0 equivalents of potassium hydroxide, 0.5 M in methanol, within a period of 5 minutes. After being refluxed for 3 to 9 hours (checked by TLC), the reaction solution was cooled to room temperature, poured into water (150 mL/mmole) and adjusted to a pH of 6 with hydrochloric acid (10% in water). The precipitate formed was isolated and recrystallized from alcohol and water. If there was no precipitate, the organic phase was removed and the aqueous phase extracted three times with ethyl acetate (in each case, with 2 mL/mmole), the combined organic phases were dried over magnesium sulfate, the solvent evaporated in a rotary evaporator and the product subsequently purified by recrystallization or the crude product was reacted as described in C2 (method D).

### EXAMPLE C1.1 (D-81687)

Educt A1: 1-(5-methoxy-1H-indole-2-yl)-ethanone

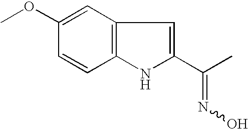

1-(5-methoxy-1H-indole-2-yl)-ethanone oxime

Melting point: 148° C.-150° C. (2-propanol)

### EXAMPLE C1.2 (D-81690)

Educt B4: 1-(5-methoxy-1H-indole-2-yl)-propane-1-one

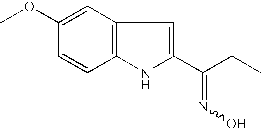

1-(5-methoxy-1H-indole-2-yl)-propane-1-one oxime

Melting point: 163° C.-165° C. (2-propanol)

### EXAMPLE C1.3 (D-70258)

Educt A2: (5-methoxy-1H-indole-2-yl)-phenyl-methanone

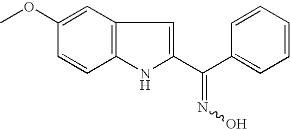

(5-methoxy-1H-indole-2-yl)-phenyl-methanone oxime

Melting point: 150° C.-152° C. (2-propanol:water=2:3)

### EXAMPLE C1.4 (D-70745)

Educt A5: (4-chlorophenyl)-(5-methoxy-1H-indole-2-yl)-methanone

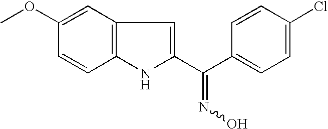

(4-chlorophenyl)-(5-methoxy-1H-indole-2-yl)-methanone oxime

Crude product (HPLC purity: 81%)

C2) Reaction of 1H-indole-2-yl-methanone oxime with N,N′-carbonyldiimidazole

To a solution of 1 equivalent of 1H-indole-2-yl-methanone oxime in tetrahydrofuran (30 mL/mmole), 1.2 equivalents of solid N,N′-carbonyldiimidazole are added and refluxed for 1 to 3 hours, and the reaction being followed by TLC. After it is cooled to room temperature, the reaction solution is poured into water (400 mL/mmole) and the precipitate formed is isolated and recrystallized from alcohol. If a precipitate is not formed, the organic phase is removed and the aqueous phase is extracted three times with ethyl acetate (in each case, with 2 mL/mmole). After the combined organic phases are dried over magnesium sulfate, the solvent is evaporated in a rotary evaporator and the product is purified by means of column chromatography on silica gel at atmospheric pressure using a 1:3 mixture of ethyl acetate and hexane.

### EXAMPLE C2.1 (D-81688)

Educt C1.1: 1-(5-methoxy-1H-indole-2-yl)-ethanone oxime

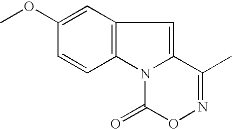

3-methyl-1,2,5-oxadiazino[4,5-a](5-methoxyindole)-6-one

Melting point: 217° C.-220° C. (2-propanol)

### EXAMPLE C2.2 (D-81691)

Educt C1.2: 1-(5-methoxy-1H-indole-2-yl)-propane-1-one oxime

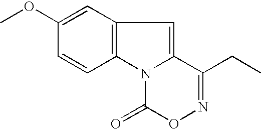

3-ethyl-1,2,5-oxadiazino[4,5-a](5-methoxyindole)-6-one

Melting point: 208° C.-212° C. (n-butanol)

### EXAMPLE C2.3 (D-70260)

Educt C1.3: (5-methoxy-1H-indole-2-yl)-phenyl-methanone oxime

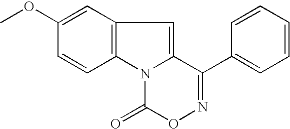

3-phenyl-1,2,5-oxadiazino[4,5a](5-methoxyindole)-6-one

Melting point: 198° C.-200° C. (n-butanol)

Method D) Direct Conversion of the 1H-indole-2-yl-methanone oxime, which has been Prepared, with N,N′-carbonyldiimidazole

The following oxa-diaza derivatives were synthesized by method C1 and were reacted further by the method of C2 without being purified.

### EXAMPLE D1 (D-81362)

Educt B1: (1H-indole-2-yl)-(2-methoxyphenyl)-methanone

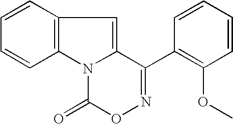

3-(2-methoxyphenyl)-1,2,5-oxadiazino[4,5a]indole-6-one

Melting point: 160° C.-162° C. (column chromatography)

### EXAMPLE D2 (D-81361)

Educt B2: (1H-indole-2-yl)-(3-methoxyphenyl)-methanone

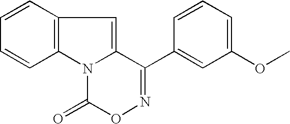

3-(3-methoxyphenyl)-1,2,5-oxadiazino[4,5a]indole-6-one

Melting point: 129° C.-130° C. (column chromatography)

### EXAMPLE D3 (D-81462)

Educt A3: (5-methoxy-1H-indole-2-yl)-3-methoxyphenyl)-methanone

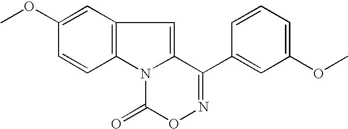

3-(3-methoxyphenyl)-1,2,5-oxadiazino[4,5a](5-methoxyindole)-6-one

Melting point: 171° C.-173° C. (ethanol)

### EXAMPLE D4 (D-70744)

Educt A5: (4-chlorophenyl)-(5-methoxy-H-indole-2-yl)-methanone

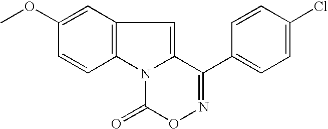

3-(4-chlorophenyl)-1,2,5-oxadiazino[4,5a](5-methoxyindole)-6-one

Melting point: 227° C.-230° C. (n-butanol)

Starting out from differently substituted indole-2-carboxylic acid derivatives, the following inventive compounds (examples No. 1 to 324) can also be synthesized by the methods C and D given above,

Educt:

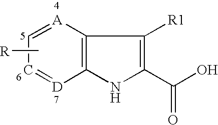

Product: (n=1, Z=N)

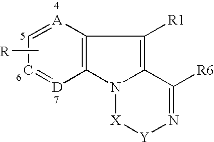

### EXAMPLES NO. 1 to 324

[symbol]

[symbol]

[symbol]

[symbol]

[symbol]

[symbol]

[symbol]

[symbol]

[symbol]

[symbol]

| | | | | | | | | |
|---|---|---|---|---|---|---|---|---|
| 317 | CH | CH | N | H | H | 2-(CH₃O)-C₆H₄ | SO₂ | O |
| 318 | CH | CH | N | H | H | 3-(CH₃O)-C₆H₄ | SO₂ | O |
| 319 | CH | CH | N | H | H | 4-(CH₃O)-C₆H₄ | SO₂ | O |
| 320 | CH | CH | N | H | H | 2,3-(CH₃O)₂-C₆H₃ | SO₂ | O |
| 321 | CH | CH | N | H | H | 2,4-(CH₃O)₂-C₆H₃ | SO₂ | O |
| 322 | CH | CH | N | H | H | 3,4-(CH₃O)₂-C₆H₃ | SO₂ | O |
| 323 | CH | CH | N | H | H | 3,5-(CH₃O)₂-C₆H₃ | SO₂ | O |
| 324 | CH | CH | N | H | H | 3,4,5-(CH₃O)₃-C₆H₂ | SO₂ | O |

Analogously, the following inventive compounds (Examples 325 to 2634) can be synthesized by the general method E:

### EXAMPLES NO. 325-654

A, C, D, R, R1, R6 and X have the meaning of the examples Nos. 1-324, which are described above, and Y in each case represents NH;

### EXAMPLES NO. 655-984

A, C, D, R, R1, R6 and X have the meaning of the examples Nos. 1-324, which are described above, and Y in each case represents N—CH₃;

### EXAMPLES NO. 985-1314

A, C, D, R, R1, R6 and X have the meaning of the examples Nos. 1-324, which are described above, and Y in each case represents N—C₂H₅;

### EXAMPLES NO. 1315-1644

A, C, D, R, R1, R6 and X have the meaning of the examples Nos. 1-324, which are ed above, and Y in each case represents N—C₆H₅;

### EXAMPLES NO. 1645-1974

A, C, D, R, R1, R6 and X have the meaning of the examples Nos. 1-324, which are described above, and Y in each case represents N-2-(CH₃O)—C₆H₄;

### EXAMPLES NO. 1975-2304

A, C, D, R, R1, R6 and X have the meaning of the examples Nos. 1-324, which are described above, and Y in each case represents N-3-(CH₃O)—C₆H₄;

### EXAMPLES NO. 2305-2634

A, C, D, R, R1, R6 and X have the meaning of the examples Nos. 1-324, which are described above, and Y in each case represents N-4-(CH₃O)—C₆H₄.

D) General Method for the Synthesis of the Inventive 1,2,4-triazino[4,5a]indole Derivatives

### EXAMPLE E1 (D-70746)

Educt A5: (4-chlorophenyl)-(5-methoxy-1H-indole-2-yl)-methanone

A suspension of 1 equivalent of the 2-acyl indole, synthesized by method A or B, in n-butanol (10 mL/mmole) was reacted with 2 equivalents of hydrazine derivative, appropriately mono-substituted, and glacial acetic acid (0.5 mL/mmole) and refluxed for 16 hours (checked by TLC). After it had cooled to room temperature, the reaction solution was poured into water to (150 mL/mmole) the organic phase was removed and the aqueous phase extracted three times with ethyl acetate (10 mL/mmole). After the combined organic phases were dried over magnesium sulfate, the solvent was evaporated carefully in a rotary evaporator and the crude product was dissolved in tetrahydrofuran (7.5 mL/mmole). This solution was treated with 1.3 equivalents of N,N′-carbonyl diimidazole and subsequently with 2.1 equivalents of sodium hydride (a 75 percent dispersion in white oil) and, after 2 hours at room temperature, refluxed for 48 hours. After it had cooled to room temperature, the reaction solution was poured into water (150 mL/mmole), the solid was isolated and the product purified by column chromatography on silica gel at atmospheric pressure using a 1:2 mixture of diethyl ether and hexane.

### EXAMPLE E1 (D-70746)

Educt A5: (4-chlorophenyl)-(5-methoxy-1H-indole-2-yl)-methanone

Reagent E1: phenylhydrazine

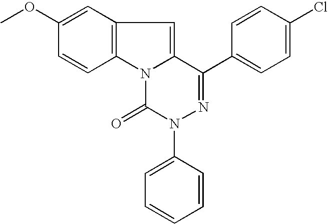

2-Phenyl-6-(4-chlorophenyl)-1,2,4-triazino[4,5a](5-methoxyindole)-3-one

Melting point: 155° C.-158° C.

F) General Method for the Synthesis of the Inventive pyrrolo[1,2-a]Indole Derivative

A solution of 1 equivalent of the 2-acyl indole in, synthesized by method A or B, N,N′-2 dimethylformamide (10 mL/mmole) was treated portionwise with 1.1 equivalents of the solid sodium hydride (a 60-75% dispersion in mineral oil) and, after five minutes of stirring act at room temperature, heated for one hour at 90° C. After the reaction solution was cooled to room temperature, 1.1 equivalents of the phenacyl halide, appropriately substituted, were added dropwise and the solution was heated once again to 90° C. for 3 to 8 hours (checked by TLC). After it had cooled to room temperature, the reaction solution was poured into water (150 mL/mmole), the precipitate formed was isolated and purified by column chromatography on silica gel at atmospheric pressure using a 1:3 mixture of diethyl ether and hexane.

### EXAMPLE f1 (D-80786)

Educt A5: (4-chlorophenyl)-(5-methoxy-1H-indole-2-yl)-methanone

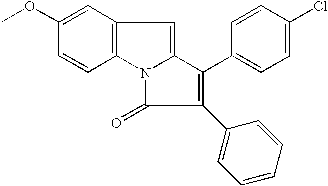

1-(4-chlorophenyl)-6-methoxy-2-phenyl-3a-aza-cyclopenta[a]indene-3-one

Melting point: 152° C.-155° C.

### Example F2 (D-80815)

Educt B6: (5-methoxy-1H-indole-2-yl)-(3-methoxyphenyl)-methanone

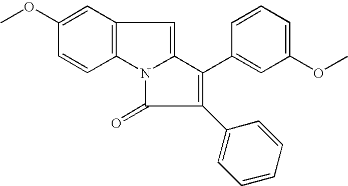

6-methoxy-1-(3-methoxyphenyl)-2-phenyl-3a-aza-cyclopenta[a]indene-3-one

Melting point: 111° C.-113° C.

### EXAMPLE F3 (D-80816)

Educt B6: (5-methoxy-1H-indole-2-yl)-(3-methoxyphenyl)-methanone

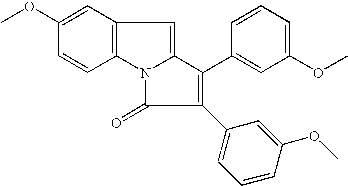

6-methoxy-1,2-bis-(3-methoxyphenyl)-3a-aza-cyclopenta[a]indene-3-one

Melting point: 112° C.-114° C.

### EXAMPLE F4 (D-80819)

Educt A4: (5-methoxy-1H-indole-2-yl)-(4-methoxyphenyl)-methanone

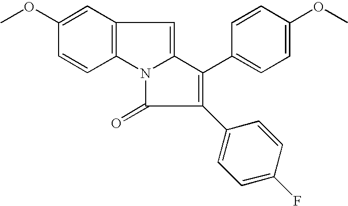

2-(4-fluorophenyl)-6-methoxy-1-(4-methoxyphenyl)-3a-aza-cyclopenta[a]indene-3-one

Melting point: 157° C.-160° C.

Starting out from differently substituted indole-2-carboxylic acid derivatives, the following inventive compounds (Examples Nos. 2635 to 3842) can be synthesized by method F given above.

Educt:

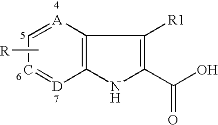

Product: (n=0, Z=C—R8)

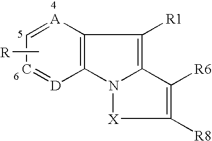

| Nr. | A | C | D | R | R1 | R6 | R8 | X |
|---|---|---|---|---|---|---|---|---|
| 2635 | CH | CH | CH | H | H | CH₃ | CH₃ | C(O) |
| 2636 | CH | CH | CH | H | H | CH₃ | C₂H₅ | C(O) |
| 2637 | CH | CH | CH | H | H | CH₃ | C₆H₅ | C(O) |

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

Analogously, the following inventive compounds (examples Nos. 3843 to 6260) can also be synthesized by the general method F:

A, C, D, R, R1, R6, R8 and X have the meanings given to them in the above examples numbered 2635 to 3842, Y in each case being Si(O).

### EXAMPLES NO. 5052-6260

A, C, D, R, R1, R6, R8 and X have the meanings given to them in the above examples numbered 2635 to 3842, Y in each case being SO₂.

Results of the Pharmacological Testing

In vitro testing in selected tumor models gave the following pharmacological activities.

### EXAMPLE 1

Antiproliferative Properties

The substances D-70260, D-70744, D-80815, D-80816 and D-80819 (Examples C2.3, D4, F2, F3 and F4) were investigated in a proliferation test (Scudiero, et al., Cancer Res., 48: 4827-33, 1987) using established tumor cell lines and their anti-proliferative activity was investigated. The test used determines the mitochondrial dehydrogenase activity and enables the cell vitality and, indirectly, the cell count to be determined. The cell lines used are the human cell lines HeLa/KB (CCL17), SK—OV-3 (HTB77), MCF-7 (HTB22) and the murine leukemia cell line L1210 (CCL219). These are very well characterized and established cell lines, which were obtained from ATCC and cultured.

The results, which are summarized in Table 1, show the very potent anti-proliferative effect of the substances D-70260, D-70744 and D-80816 (Examples C2.3, D4 and F3). On the other hand, the structurally related compounds D-80815 and D-80819 (Examples F2 and F4) do not show a significant effect. There are therefore defined structure-activity relationships.

TABLE 1
The anti-proliferative activity of different derivatives
in the XTT cytotoxicity test on the cell lines HeLa/KB,
SK-OV-3, MCF-7 and L1210. The percentage inhibition at a
concentration of 3.16 μg/mL is given.

| | | Concen- | | | | |
|---|---|---|---|---|---|---|
| | Code | tration | | | | L |
| Example | No. | (μg/mL) | KB/HeLa | SK-OV-3 | MCF-7 | 1210 |
| C2.3 | D-70260 | 3.16 | 90.7 | 68.9 | 4.1 | 91.3 |
| D4 | D-70744 | 3.16 | 74.6 | 55.2 | 1.9 | 77.1 |
| F2 | D-80815 | 3.16 | 1.7 | 0 | 8.1 | 0 |
| F3 | D-80816 | 3.16 | 91.7 | 66.1 | 18.2 | 94.7 |
| F4 | D-80819 | 3.16 | 0 | 6.6 | 0 | 21.8 |

[image]

### EXAMPLE 2

Effect of D-80816 (example F3) in the Hollow Fiber Model in vivo

In order to determine the availability and effectiveness in the animal model (nude mouse), the cell lines HeLa/KB, MCF-7 and L1210 were cultured in hollow fibers, which are implanted i.p. or s.c. (Hollingshead et al., Life Sciences 57, 131-41, 1995). The test substance D-80816 is administered four times c.p. in a dose of 100 mg/kg. At the end of the therapy on day 5, the fibers are explanted and the cell vitality of the tumor cells obtained is determined by means of the XTT assay. For D-80816, there is a maximum inhibition of 100% for all the cell lines and implantation cites and a general toxicity of LD₅₀>1000 mg/kg (i.p.).

TABLE 2
In-vivo activity of D-80816 (dose: 4× 100 mg/kg i.p.) in the hollow
fiber test with the tumor cell lines HeLa/KB, MCF-7 and L1210.

| | | | Proliferation Inhibition | |
|---|---|---|---|---|
| | | LD 50 | in Hollow Fibers | |
| Example | Code No. | (mg/kg) | (%) | |
| F3 | D-80816 | >100 mg/kg i.p. | KB (i.p.) | 100% |
| | | | KB (s.c.) | 100% |
| | | | L1210 (i.p.) | 100% |
| | | | L1210 (s.c.) | 100% |
| | | | MCF7 (i.p.) | 100% |
| | | | MCF7 (s.c.) | 100% |

### EXAMPLE 3

Cell Cycle-Specific Activity of D-80816 in the RKOp27 Model

As a model for investigating the cell cycle-specific activity, the RKOp27 cell system was used (M. Schmidt et al. Oncogene 19 (20) 2423-9, 2000). The RKO is a human, colon carcinoma line, in which the cell cycle inhibitor p27^{Kip1} is induced to express by means of the Ecdyson expression system and leads to a cell cycle arrest specifically in G1 (FIG. 2). A substance with non-specific activity inhibits the proliferation independently of whether the RKO cell in G1 is arrested or not. On the other hand, cell cycle-specific substances, such as tubulin inhibitors, are cytotoxic only when the cells are not arrested and the cells pass through the cycle. D-80816 shows a cell cycle-specific activity here, that is, a concentration-dependent anti-proliferative effect can be measured only in cells, which are not induced and cannot be measured in cells, which are arrested in G1 of the cell cycle (FIG. 3). Therefore, a defined, molecular, activity mechanism of D-80816 and its derivatives must be assumed.

[image]

[image]

### DESCRIPTION OF THE METHODS USED

XTT Test for Cellular Dehydrogenase Activity

The adherent, growing, tumor cell lines HeLa/KB, SK—OV-3, MCF-7, L1210 and RKO were cultured under standard conditions in an incubator with gas inlet at 37° C., 5% carbon dioxide and 95% relative humidity. On day 1 of the experiment, the cells were detached with trypsin/EDTA and palletized by centrifuging. Subsequently, the cell pellet is re-suspended in the respective culture medium in the appropriate cell count and transferred into a 96-well microtiter plate. The plates are then cultured overnight in the incubator with gas inlet. The test substances are used as a 10 mM stock solution in DMSO and diluted on the second day of the experiment with culture medium to the appropriate concentrations. The substances in the culture medium are then added to the cells and incubated for 45 hours in the incubator with gas inlet. The cells, which were not treated with test substances, were used as controls. For the XTT-Assay, 1 mg/mL of XTT (sodium 3′-[1-(phenylaminocarbonyl)-3,4-tetrazolium]-bis(4-methoxy-6-nitro)benzenesulfonic acid) in RPMI-1640 medium was dissolved without phenol red. Additionally, a 0.383 mg/mL PMS (N-methyl dibenzopyrazine methyl sulfate) solution in phosphate buffer cell solution (PBS) was prepared. On the fourth day of the experiment, 75 μL/well of XTT-PMS mixture was pipetted onto the cell plates, which had meanwhile been incubated for 45 hours with the test substances. For this purposes, the XTT solution is mixed with the PMS solution in a ratio of 50:1 (v:v) shortly before use. Subsequently the cell plates are incubated in the incubator with gas inlet for a further three hours and the optical density (OD_{490 nm}) is determined in a photometer.

The percentage inhibition relative to the control is calculated by means of the OD_{490 nm}, which has been determined, and the activity is plotted against the log of a concentration. The IC₅₀ is calculated from the concentration-activity curve by means of a regression analysis using the Graphpad program.

Determination of the Anti-Proliferative Activity in the Hollow Fiber Model in vivo

The tumor cells lines HeLa/KB, MCF-7 and L1210 are added to polyvinylidene fluoride hollow fibers (5×10⁶ cells/mL) and transplanted into the physiological compartments of the nude mouse (intraperitoneally, i.p., or subcutaneously, s.c.). A total of six hollow fibers (3 i.p. and 3 s.c.) with the respective tumor cell lines is transplanted into each experimental animal. One group of six animals is treated with the test substance (i.p. once daily for a total of 4 days) (ok?). The animals, which have been treated only with the solvent tylose, acted as control. The hollow fibers are explanted one day after the last application of substance. The proportion of metabolism-active, vital cells is determined for each hollow fiber by means of the XTT assay (see above). From this, the anti-tumor activity of the test substance is determined as the percent inhibition relative to the control.

Cell Cycle Analysis by Means of the RKOp27 Model

The assay is carried out in 96-well plates. The growth of the cells is arrested completely by the expression of p27^{kip1}, which can be induced. However, the cells do not die. By comparing the effectiveness of induced and not-induced cells, conclusions can be drawn concerning the mechanism of action (cell cycle specificity) of the therapeutic agents. Cells, which have not been induced, were disseminated in an approximately 4 times higher cell count than uninduced cells, since there no longer is a division during the assay (2×10⁴ cells/well induced, about 0.6×10⁴ cells/well not induced). The controls are untreated cells (± induction). The induction is carried out with 3 μM of muristerone A. The cells are exposed on the first day (± muristerone A) and incubated for 24 hours at 37° C. The test substance is added on the second day (control DMSO) and the incubation is continued for a further 48 hours at 37° C., before the standard XTT assay is carried out (see above).

The inventive compounds can be used as pharmaceuticals for the treatment of diseases, especially of tumor diseases, in mammals and especially in man.

The inventive compounds can be administered in suitable forms orally, topically or parenterally (i.m., i.v., s.c.).

The following are mentioned as suitable forms of administration:

### EXAMPLE I

Tablet with 50 mg of Active Ingredient

Composition:

| | |
|---|---|
| (1) active ingredient | 50.0 mg |
| (2) lactose | 98.0 mg |
| (3) cornstarch | 50.0 mg |
| (4) polyvinylpyrrolidone | 15.0 mg |
| (5) magnesium stearate | 2.0 mg |
| Total: | 215.0 mg |

Preparation:

(1), (2) and (3) are mixed and granulated with an aqueous solution of (4). (5) is added to the dried granulate. Tablets are pressed from this mixture.

### EXAMPLE II

Capsule with 50 mg of Active Ingredient

Composition:

| | |
|---|---|
| (1) active ingredient | 50.0 mg |
| (2) cornstarch dried | 58.0 mg |
| (3) pulverized lactose | 50.0 mg |
| (5) magnesium stearate | 2.0 mg |
| Total: | 160.0 mg |

Preparation:

(1) and (3) are ground together. The mixture of (2) and (4) is added to the ground material with intensive mixing. This powder mixture is filled into hard gelatin capsules (size 3), using a capsule-filling machine.

1. Compounds of the general formula I 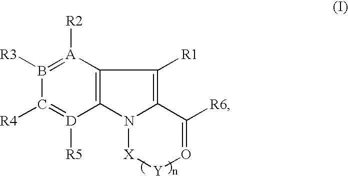
in which
R1 represents hydrogen, unsubstituted or fully or partly substituted, identically or differently, (C6-C14)-aryl, unsubstituted or fully or partly substituted, identically or differently, (C1-C13)-heteroaryl, having at least one to four N, NH, O and/or S as ring elements, unsubstituted or fully or partly substituted, identically or differently, (C3-C8)-cycloalkyl, or unsubstituted or fully or partly substituted, identically or differently (C1-C20)-alkyl,
A, B, C, or D, independently of one another, represents a carbon atom or a nitrogen atom, substituted with R2-R5,
R2, R3, R4 and R5, independently of one another represent a free electron pair (when A, B, C or D represents nitrogen), hydrogen, halogen, cyano, nitro, hydroxy, linear or branched (C1-C6)-alkyl, linear or branched (C1-C6)-alkyl, substituted with one or more halogen atoms, linear or branched (C1-C6)-alkoxy, substituted with one or more halogen atoms, linear or branched (C1-C6)-alkoxy, linear or branched (C1-C6)-alkylenedioxy, (C1-C6)-alkylcarbonyloxy, (C1-C6)-alkoxycarbonyloxy, (C1-C6)-alkylthio, (C1-C6)-alkylsulfinyl, (C1-C6)-alkylsulfonyl, carboxy, carboxy(C1-C6)-alkylester, carboxamide, N-(C1-C6)-alkylcarboxamide, N,N-di-(C1-C6)-alkylcarboxamide, (C1-C6)-alkoxy-(C1-C6)-alkyl, amino, mono-(C1-C6)-alkylamino, di-(C1-C6)-alkylamino, the two (C1-C6) groups together being able to form a ring, which optionally has one or more NH, N—(C1-C6)-alkyl, O or S, (C6-C14)-aryl, (C6-C14)-aryloxy, (C6-C14)-aryl-(C1-C6)-alkyl, (C6-C14)-aryl-(C1-C6)-alkoxy-(C1-C6)-alkyl, (C1-C6)-alkylcarbonyl, (C1-C6)-alkoxycarbonyl, hydroxy, in which two directly adjacent groups can be linked to one another;
R6 represents unsubstituted or fully or partly substituted, identically or differently, (C6-C14)-aryl, unsubstituted or fully or partly substituted, identically or differently, (C1-C13)-heteroaryl, having at least one to four N, NH, O and/or S as ring elements, unsubstituted or fully or partly substituted, identically or differently, (C3-C8)-cycloaklyl, unsubstituted or fully or partly substituted, identically or differently, linear or branched (C1-C20)-alkyl, the identical or different substituents being selected from the group comprising hydrogen, fluorine, chlorine, bromine, iodine, cyano, nitro, hydroxy, (C1-C6)-alkyl, (C1-C6)-alkoxy, carboxy, (C1-C6)-alkyl, substituted, identically or differently, with one or more halogen atoms, (C1-C6)-alkoxy, substituted identically or differently with one or more halogen atoms, linear or branched (C2-C6)-alkenyl, linear or branched (C2-C6)-alkinyl, (C3-C8)-cycloalkyl, linear or branched (C1-C6)-alkoxy, linear or branched (C1-C6)-alkylenedioxy, (C1-C6)-alkoxy-(C1-C6)-alkyl, (C6-C14)-aryl, (C6-C14)-aryl-(C1-C6)-alkyl, (C6-C14)-aryl-(C1-C4)-alkoxy-(C1-C6)-alkyl;
X represents carbonyl-(C═O), sulfoxide-(S═O) or the sulfonyl group (SO₂);
Y represents an oxygen atom or a nitrogen atom (NR7), substituted by the R7 group, in which
R7 represents unsubstituted or fully or partly substituted, identically or differently, (C6-C14)-aryl, unsubstituted or fully or partly substituted, identically or differently, (C1-C13)-heteroaryl, having at least one to four N, NH, O and/or S as ring elements, (C3-C8)-cycloalkyl, unsubstituted or fully or partly substituted, identically or differently, (C1-C20)-alkyl, which may be linear or branched, wherein the identical or different substituents are selected from the group comprising hydrogen, fluorine, chlorine, bromine, iodine, cyano, nitro, hydroxy, (C1-C6)-alkyl, (C1-C6)-alkoxy, carboxy, (C1-C6)-alkyl, substituted identically or differently with one or more halogen atoms, (C1-C6)-alkoxy, substituted identically or differently with one or more halogen atoms, linear or branched (C2-C6)-alkenyl, linear or branched (C2-C6)-alkinyl, (C3-C8)-cycloalkyl, linear or branched (C1-C6)-alkoxy, linear or branched (C1-C6)-alkylenedioxy, (C1-C6)-alkoxy-(C1-C6)-alkyl, linear or branched mono-(C1-C6)-alkylamino, linear or branched di-(C1-C6)-alkylamino, wherein the two (C1-C4) groups together may form a ring, which optionally has one or more NH, N—(C1-C6)-alkyl, O or S, (C6-C14)-aryl, (C6-C14)-aryl-(C1-C6)-alkyl, (C6-C14)-aryl-(C1-C4)-alkoxy-(C1-C6)-alkyl, (C1-C6)-alkylcarbonyl, (C1-C6)-alkoxycarbonyl;
n is 0 or 1, with the proviso that, when n=0,
Z represents a carbon atom (C—R8), substituted with the R8 group wherein
R8 represents unsubstituted or fully or partly substituted, identically or differently, (C6-C14)-aryl, unsubstituted or fully or partly substituted, identically or differently (C1-C13)-heteroaryl, which has at least one to four N, NH, O and/or S as ring elements, unsubstituted or fully or partly substituted, identically or differently, (C3-C8)-cycloalkyl, unsubstituted or fully or partly substituted, identically or differently, linear or branched, (C1-C20)-alkyl, the identical or different substituents being selected from the group comprising hydrogen, fluorine, chlorine, bromine, iodine, cyano, nitro, hydroxy, (C1-C6)-alkyl, (C1-C6)-alkoxy, carboxy, (C1-C6)-alkyl, which is substituted identically or differently by one or more halogen atoms, (C1-C6)-alkoxy, which is substituted identically or differently by one or more halogen atoms, linear or branched (C2-C6)-alkenyl, linear or branched (C2-C6)-alkinyl, (C3-C8)-cycloalkyl, linear or branched (C1-C6)-alkoxy, linear or branched (C1-C6)-alkylenedioxy, linear or branched (C1-C6)-alkylthio, (C1-C4)-alkylsulfinyl, (C1-C4)-alkylsulfonyl, (C6-C14)-arylthio, (C6-C14)-arylsulfinyl, (C6-C14)-arylsulfonyl, (C1-C6)-alkoxy-(C1-C6)-alkyl, linear or branched mono-(C1-C6)-alkylamino, linear or branched di-(C1-C6)-alkylamino, wherein the two (C1-C4) groups together can form a ring, which optionally has one or more NH, N—(C1-C6)-alkyl, O or S, (C6-C14)-aryl, (C6-C14)-aryloxy, (C6-C14)-aryl-(C1-C6)-alkyl, (C6-C14)-aryl-(C1-C4)-alkoxy-(C1-C6)-alkyl, (C1-C6)-alkylcarbonyl, (C1-C6)-alkoxycarbonyl, linear or branched mono-N—(C1-C6)-alkylcarbonylamino, linear or branched di-N,N—(C1-C6)-alkylcarbonylamino, linear or branched di-N,N—(C1-C6)-alkoxycarbonylamino, linear or branched mono-N—(C1-C6)-alkoxycarbonylamino, linear or branched N—(C1-C6)-alkylcarbonylamino-N—(C1-C6)-alkylamino, linear or branched N—(C1-C6)-alkoxycarbonylamino-N—(C1-C6)-alkylamino,
and, when n=1,
Z represents a nitrogen atom;
their tautomers, stereo isomers, mixtures and pharmaceutically tolerated salts.

2. The compound of claim 1, wherein R1 represents hydrogen, R2, R3, R4 and R5 independently of one another represent hydrogen or halogen or (C1-C6)-alkoxy, R6 represents unsubstituted or fully or partly substituted, identically or differently, linear or branched (C1-C20)-alkyl or unsubstituted or fully or partly substituted, identically or differently, (C6-C14)-aryl, which is substituted with (C1-C6)-alkoxy and halogen, and Y represents oxygen or the N—R7 group, in which R7 represents unsubstituted or fully or partly substituted, identically or differently, (C6-C14)-aryl, X is carbonyl (C═O), Z is a nitrogen atom and n=1.

3. The compound of claim 1, wherein R1 represents hydrogen, R2, R3, R4 and R5 independently of one another represent hydrogen, halogen or (C1-C6)-alkoxy, R6 represents unsubstituted or fully or partly substituted, identically or differently, linear or branched (C1-C20)-alkyl or unsubstituted or fully or partly substituted, identically or differently, (C6-C14)-aryl, which is substituted with (C1-C6)-alkoxy and halogen, n=0, Z represents the C—R8 group, in which R8 represents unsubstituted or fully or partly substituted, identically or differently, (C6-C14)-aryl substituted with (C1-C6)-alkoxy and halogen, and X represents carbonyl (C═O), I made available.

4. The compound of one of the claims of 1 to 3 for use as a pharmaceutical.

5. Use of a compound of one of the claims of 1 to 3 for controlling tumor diseases in mammals, especially in man.

6. A pharmaceutical with at least one compound of claims 1 to 3 together with conventional pharmaceutical auxiliary materials, diluents and/or carrier materials.

7. A method for the synthesis for compounds of the general formula I 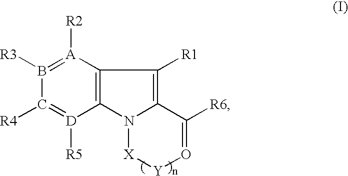
wherein A, B, C, D, R1, R2, R3, R4, R5, R6, X, Y, Z and n have the meaning in claim 1, characterized by the reaction of the ketone of the general formula 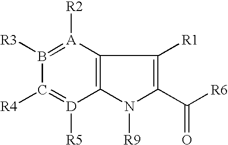
wherein A, B, C, D, R1, R2, R3, R4, R5 and R6 have the meanings given above and R9 represents hydrogen or a suitable leaving group, such as unsubstituted or fully or partly substituted, identically or differently, linear or branched (C1-C6)-alkylcarbonyl, linear or branched (C1-C6)-alkoxycarbonyl, substituted (C6-C14)-aryl-(C1)-alkyl, linear or branched (C1-C6)-alkylsulfonyl and (C6-C14)-arylsulfonyl, which is unsubstituted or fully substituted with (C1-C6)-alkyl,
1.) (if n=1) with
1.1) (if Y=oxygen) hydroxylamine or
1.2) (if Y═NR7), a hydrazine derivative H2N—NH—R7, wherein R7 as the meaning given above and reaction of the product, so obtained, with an activated carbonyl, sulfoxide or sulfonyl derivative with ring closure and
or
2.) (if n=0) with a phenylacetic acid derivative X1-CO—CH2-R8, wherein X1 represents a suitable leaving group, such as halogen or (C1-C6)-alkoxy and R8 has the meaning given above, and subsequent ring closure in the presence of a base, is made available.
